(19) United States
(12) Patent Application Publication
(10) Pub. No.: US 2022/0354432 A1
(43) Pub. Date: Nov. 10, 2022

(54) CARDIAC HEALTH ASSESSMENT SYSTEMS AND METHODS

(71) Applicant: Cortery AB, Domsten (SE)

(72) Inventor: Filip Ludwig Peters, Domsten (SE)

(21) Appl. No.: 17/315,261

(22) Filed: May 7, 2021

(51) Int. Cl. A61B 5/00; G16H 40/67; G16H 50/70; G16H 10/65; G16H 50/20; G16H 50/30; G06T 7/00; A61B 7/04; A61B 5/11; A61B 5/021; A61B 5/0205

(52) U.S. Cl. CPC A61B 5/6898; G16H 40/67; G16H 50/70; G16H 10/65; G16H 50/20; G16H 50/30; G06T 7/0012; A61B 7/04; A61B 5/1102; A61B 5/7264; A61B 5/02108; A61B 5/7435; A61B 5/7475; A61B 5/7278; A61B 5/7282; A61B 5/4878; A61B 5/0205; A61B 5/7275; G06F 3/044

(57) ABSTRACT

A cardiac health assessment system includes a memory, a circuit board, and a touchscreen controller integrated into a handheld electronic device (HED). The memory stores a classification model, a regression model, and instructions about a cardiac monitoring application. The circuit board includes a microphonic sensor, an Inertial Measurement Unit (IMU) sensor, a camera sensor, and a processor. The microphonic sensor captures cardiac sound wave signals indicative of the cardiac health of a user. The IMU sensor captures seismic signals indicative of the cardiac health of the user. The camera sensor enables visual data collection of tissue and photoplethysmography. The processor is configured to: execute the instructions, display commands to position the HED against the chest of the user, detect abnormal heart activity by deploying the classification model, and estimate intracardiac pressure by deploying the regression model. The touchscreen controller displays cardiac diagnostic information.

### BACKGROUND

### Technical Field

The inventive subject matter disclosed herein is generally directed towards a portable handheld electronic device (HED) for cardiac health assessment, and more particularly, but not limited to, cardiac health assessment systems and methods having various sensors configured within the HED.

### Description of the Related Art

Optimizing medical surveillance and therapy for patients with symptomatic heart failure (HF) and heart disease is important in helping avoid acute decompensations and complications. For many HF patients, worsening heart health is assessed by clinical evaluation of body weight or jugular venous pressure. Changes in these parameters however appear late in the process of HF decompensation and are relatively unreliable indicators. Daily measurement of body weight for example, has low sensitivity for exacerbating HF. Remote monitoring techniques, on the other hand, have shown efficacy in reducing HF re-hospitalizations and mortality rates, particularly when invasive hemodynamic data are acquired.

The release of CardioMEMS, a Food and Drug Administration-approved remote wireless pulmonary artery pressure (PAP) monitoring system, has demonstrated major progress in acute decompensated HF event prevention, showing for the first time efficacy in reducing heart failure patient hospitalizations by remote monitoring of pressures in the cardiovascular system. Several challenges exist however with these invasive intracardiac pressure monitoring technologies that need to be addressed.

These tools are currently limited to measurement of pulmonary arterial pressure (PAP) and monitoring of the left chambers has not been available for HF patients. Since approximately 90% of patients admitted to the hospital for HF have pulmonary congestion related to elevated left atrial pressure (LAP), this may significantly impact the efficacy of these tools. In cases where post-capillary pulmonary hypertension is present, PAP sensors are also less indicative of congestion, a common feature of HF with preserved ejection fraction (HFpEF). Left-sided filling pressures may therefore not be accurately estimated in cases of increased pulmonary resistance, which occurs in more than 50% of patients with advanced HF.

Existing solutions can be invasive and there is concern about their cost efficiency at current prices. They can also fail to identify precipitating factors of heart failure hospitalizations, for example mitral regurgitation, myocardial ischemia, atrial arrhythmias, uncontrolled hypertension, and the like.

Changes in the healthcare model towards reducing inpatient hospitalizations could have a significant impact on HF-related costs and quality of life. Wireless and remote monitoring has the potential to become an essential part of the health management process in patients with HF. The emergence of smartphone and wrist-based sensing devices has led to potential improvements in access and ease of use, with the advancements of machine learning methods and computer processing capabilities making sophisticated analysis possible from many smartphones. Non-invasive sensor devices may provide an easy-to-use and objective alternative to existing remote HF monitoring that typically require invasive procedures.

There are various systems and methods exist that describe predicting heart diseases. For example, US patent publication 20150057512A1 to Kapoor discusses a system for acquiring electrical footprint of the heart, electrocardiogram (EKG or ECG), heart sound, heart rate, nasal airflow, and pulse oximetry with the system being incorporated into a mobile device accessory. The ECG and heart sound signals are conveniently acquired and transmitted to a server via the mobile device, offering accurate cardiovascular analysis, and sleep disorder breathing indication.

U.S. Pat. No. 8,509,882 issued to Albert, et al. discloses a personal monitoring device having a sensor assembly configured to sense physiological signals upon contact with a user's skin. The sensor assembly produces electrical signals representing the sensed physiological signals. A converter assembly, integrated with, and electrically connected to the sensor assembly, converts the electrical signals generated by the sensor assembly to a frequency modulated physiological audio signal has a carrier frequency in the range of from about 6 kHz to about 20 kHz.

Prior art approaches for assessing the cardiac health of patients in real-time several can have problems such as portability, effectiveness, accuracy, and cost among other items. Machine learning (ML) in combination with portable devices incorporating non-invasive sensor technologies has the potential to save the lives of current and future heart patients.

Therefore, there is a need for a cardiac health assessment system that leverages a unique combination of sensor technologies and machine learning algorithms to provide an affordable and portable device to assess cardiac health. Additionally, there is a need for a product that can accurately identify several cardiac conditions in order to facilitate preventive healthcare for heart failure patients. There is therefore a need for a cardiac health assessment system that is non-invasive and can allow for both affordable and effective remote heart failure monitoring.

In view of the above, there is a long-felt need in the healthcare industry to address these deficiencies and inadequacies.

The approaches described in this section are approaches that could be pursued, but these are not necessarily approaches that have been previously conceived or pursued. Therefore, unless otherwise indicated, it should not be assumed that any of the approaches described in this section qualify as prior art merely by virtue of their inclusion in this section.

### SUMMARY

Cardiac health assessment systems and methods are provided, as shown in and/or described in connection with at least one of the figures.

One aspect of the present disclosure relates to a cardiac health assessment system that includes a memory, a circuit board, and a touchscreen controller that is integrated into a HED. The memory stores a classification model, a regression model, and a plurality of instructions about a cardiac monitoring application. The circuit board includes a microphonic sensor, an Inertial Measurement Unit (IMU) sensor, a camera sensor, and a processor. The microphonic sensor captures cardiac sound wave signals indicative of the cardiac health of a user. The Inertial Measurement Unit (IMU) sensor captures seismic signals indicative of the cardiac health of the user. The camera sensor enables visual data collection of tissue and photoplethysmography. The processor is configured to: execute the plurality of instructions about the cardiac monitoring application; display one or more commands to position the HED against the chest of the user, detect an abnormal heart activity arising from a plurality of parameters by deploying the classification model; and estimate intracardiac pressure by deploying the regression model. The touchscreen controller displays cardiac diagnostic information derived from the cardiac sound wave signals received from the microphonic sensor.

In an embodiment, the tissue's color and structure may be visually analyzed with the camera and machine learning based image analysis. Heart failure may result in organs, muscles and other types of tissue not getting enough oxygen which may be result in visual abnormalities such as tissue color and/or structure.

In an embodiment, the cardiac health assessment system includes a temperature sensor. Heart chamber volumes correlate with intracardiac pressures and variations in surface skin temperature may arise from differences in the volume of one or more of the patient's heart chambers arising from changes in intracardiac pressure. Changes in tissue from reduced oxygen levels may furthermore cause changes in tissue temperature. Reduced blood flow to organs may furthermore cause abnormal changes in temperature, observable on the surface.

In an embodiment, the cardiac health assessment system includes a patient risk stratification model to estimate future adverse patient health event risks. Such a model may comprise of the patient's data collected through the cardiac health assessment system and may be trained on ex-post patient outcomes. Such outcomes may include but are not limited to hospitalizations, mortality rates, morbidity rates and/or general patient health assessments.

In an embodiment, the cardiac health assessment system includes a battery configured to supply electrical power to the circuit board.

In an embodiment, the HED has a curved shape adapted to fit firmly on the user's chest.

In an embodiment, the plurality of parameters comprising hypertension, atrial fibrillation, heart arrythmias, coronary artery disease, ischemic cardiomyopathy, aortic stenosis, aortic regurgitation, mitral stenosis, and/or mitral regurgitation.

In an embodiment, the processor triggers a message transmission to a computing device of a healthcare professional upon detection of high severity of heart disease based on cardiac data signals captured by the HED.

In an embodiment, the processor identifies a plurality of unique physiological markers of the user based on cardiac data signals captured by the HED.

In an embodiment, the processor is configured to display a plurality of instructions regarding the management of the user's disease.

In an embodiment, the classification model and the regression model are trained with the use of intracardiac pressure data measured from a catheter and/or an invasive sensor as a gold standard.

In an embodiment, the cardiac monitoring application is based on one or more operating systems comprising one or more of the following: Amazon Fire®, One UI®, Librem®, EMUI®, Android®, and iOS®.

In an embodiment, the cardiac health assessment system includes a wearable device worn by the user to obtain physiological data of the user and transmit it to the HED over a network.

In various embodiments, the circuit board includes one or more diaphragms to enhance the cardiac audio signals captured by the microphonic sensor.

In an embodiment, the circuit board includes a soundwave transducer to transmit soundwaves into the body of the user to deflect soundwaves arising from a plurality of physiological processes comprising intracardiac blood pressure and heart movements and return sound wave data to be analyzed with the classification model and the regression model. Said soundwaves may be pulsed and vary in frequency to improve penetrability.

In an embodiment, the circuit board includes an infrasound transducer for transmitting low-frequency waves into the body of the user to deflect the physiological processes and return wave data to be analyzed with the classification model and/or the regression model. Said soundwaves may be pulsed and vary in frequency to improve penetrability.

In an embodiment, the circuit board includes a magnetometer to detect abnormal traces of ferromagnetic levels in the blood of the user.

An aspect of the present disclosure relates to a method for cardiac health assessment. The method includes a step of integrating a memory, a circuit board, and a touchscreen controller in a HED. The method includes a step of storing, in a memory, a classification model, a regression model, and a plurality of instructions about a cardiac monitoring application. The circuit board comprises a microphonic sensor; an IMU sensor, a camera sensor, a processor, a diaphragm; an ultrasound transducer; an infrasound transducer, and a magnetometer. The method includes a step of capturing, by the microphonic sensor, cardiac sound wave signals indicative of the cardiac health of a user. The method includes a step of capturing, by the IMU sensor, seismic signals indicative of the cardiac health of the user. The method includes a step of performing, by the camera sensor, visual data collection of tissue and photoplethysmography. The method includes a step of executing, by the processor, the instructions about the cardiac monitoring application. The method includes a step of displaying, by the processor, one or more commands to position the HED against the chest of the user. The method includes a step of detecting, by the processor, an abnormal heart activity arising from a plurality of parameters by deploying the classification model. The method includes a step of estimating, by the processor, intracardiac pressure by deploying the regression model. The method includes a step of displaying, by the touchscreen controller, cardiac diagnostic information derived from the cardiac signals received from the sensors. The method further comprising a step of supplying, by a battery, electrical power to the circuit board.

The method further comprises a step of obtaining, by a wearable device worn by the user, physiological data of the user and transmit the physiological data to the HED over a network.

In an embodiment, the physiological data of the user obtained from the wearable device is used to estimate the likelihood of the patient adhering to their medicine by analyzing the patient's historical proximity to the medicine container that the patient uses to store its medicine.

In an embodiment, the magnetometer detects abnormal traces of ferromagnetic levels in the blood of the patient/user. Detection of such abnormal traces may aid in determining whether the patient is adhering to their medication if there are ferromagnetic compounds present in the patient's medicine.

In an embodiment, the cardiac health assessment system includes a lens configured to envelop the camera of the HED. The lens may be configured to cover all or a portion of the camera of the HED. In some of these embodiments, the lens is configured to block all or a portion of the external light while light is directed onto and into the skin of the patient and images and/or video of the skin and the associated features of the skin are simultaneously captured. In some embodiments, video, in addition to or instead of, one or more images may be recorded by the camera.

In an embodiment, the cardiac health assessment may include a flash for illuminating skin tissue during image acquisition by the camera.

In an embodiment, the camera sensor may include one or more lights that can emit one or more flashes of light that can be directed to the patient's skin.

In an embodiment, the color of the flash may be adjusted according to the patient's tissue color to optimize light penetration and to illuminate certain physiological properties. For example, in some embodiments, certain skin colors may require different light wavelengths to enable visualization and certain physiological properties. In some embodiments, visualizing veins may require a particular light of flash to optimize illumination.

In an embodiment, the cardiac health assessment may include multiple microphonic sensors to enable improved noise cancellation and segmentation.

In an embodiment, the cardiac health assessment may include multiple IMU sensors to enable improved noise cancellation and segmentation.

In an embodiment the diaphragm may comprise a piezoelectric material.

In an embodiment the diaphragm may comprise piezoelectric ceramic disks adhered to metal plates constructed of brass or nickel-alloy.

In an embodiment, cardiac health may furthermore be assessed after the user is instructed to place the HED against their thoracic cage. In many embodiments, lung fluid levels may be assessed to determine worsening heart failure resulting from increased congestion in the heart. Congestive heart failure and left-sided congestion may cause pulmonary edema if the heart is not able to pump blood efficiently, for example when blood backs up into the veins that function to take blood through the lungs. Pressure in such blood vessels may increase and fluid is pushed into the air spaces (alveoli) in the lungs. Changes in fluid levels in lungs will affect how soundwaves and seismic waves travel through lungs and deviations from normal lung fluid levels may indicate increases in intracardiac pressure.

In an embodiment, pulmonary health may be assessed after the user is instructed to place the cardiac health assessment system against their thoracic cage and the system is put into place. Chronic obstructive pulmonary disease is a common precipitating factor in heart failure and an important health condition to analyze as it pertains to preventive heart failure care. Pulmonary embolism (PE) is a common complication in heart failure patients and non-invasive identification of acute PE may arise from deviations in lung data that arise from free-floating thrombus in the right heart or pulmonary artery. Thus, enabling pulmonary health assessment can improve the efficacy in helping reduce heart failure hospitalizations. The present pulmonary health assessment systems explore the presence of abnormalities and deviations from a typical patient's normal lung function. For example, the pulmonary health assessment device may be used to observe abnormal masses or nodules using the non-invasive sensor technologies. Small lesions in a patient's lungs can also be indicative of pulmonary health conditions pertaining to cancer and/or other diseases. Physical symptoms arising from pulmonary health deterioration, for example in cases where a patient has a cough and/or is producing sputum, may be discovered through deviations in seismic and/or audio waveforms. Inflammation in the lungs may also be discovered using the technologies described herein through deviations from normal audio, seismic, photoplethysmographic and/or electrocardiographic readings of lung function. Scarring of the lungs can leave lasting marks on a patient's pulmonary health that may be indicative of pulmonary fibrosis and may be identified by certain bilateral fine crackles, for instance a “Velcro-like” sound or another similar distinctive sound. The progression of certain types of pulmonary health conditions may also be assessed by analyzing the severity of the deviations from those conditions found in a normal lung.

In an embodiment, thrombotic risk may be assessed after the user is instructed to place the cardiac health assessment system against the skin of a part of the user's lower limb region and the system is placed into that region. Patients with heart failure are particularly vulnerable to the development of venous thromboembolism (VTE) and its related complications. This can include deep vein thrombosis which occurs when blood flowing within a vein becomes clotted and a thrombus starts building up and occluding regular blood flow within the vein. Such a thrombus may become enlarged and be released into the rest of the cardiac system resulting in an embolus which may result in a pulmonary embolism that can potentially be fatal. Such internal venous dynamics can lead to indications encompassed in signals that can be derived from a number of sensors, including but not limited to IMU sensors, photoplethysmographic and acoustic sensors. When clots build up and/or are released, the internal dynamics of the vein changes, and a waveform typically associated with normal blood flow may temporarily be disrupted. Normal blood flow can be expected to be represented by a stable and recurring wavelike form. These types of blood flow disruptions may not be easily identifiable so the embodiments may utilize machine learning methods to aid in the detection of small perturbations and variance in venous blood flow. These intervals may reveal underlying hemodynamic problems thus representing another reason why unique data points relating to IMU and/or other sensor technology through the extended monitoring using one or more physiological sensors can be important for understanding the general health of a patient.

One advantage of the present inventive subject matter is to provide a real-time diagnostic mechanism for heart disease and heart failure.

Another advantage of the present inventive subject matter is that it provides a cardiac monitoring application that incorporates reminders, nudges, and notifications to help a heart patient track the risk of heart diseases and worsening heart failure.

Another advantage of the present inventive subject matter is that it provides a telehealth mechanism to facilitate the user to interact with a clinician or get on a video call with the clinician.

Another advantage of the present inventive subject matter is that the telehealth mechanism allows the user to interact with a medical chatbot to get information and provide feedback.

Another advantage of the present inventive subject matter is to provide remote patient monitoring and management of heart diseases.

Another advantage of the present inventive subject matter is to provide treatment and medication adjustments based on the results assessed by the HED.

These features and advantages of the present disclosure may be appreciated by reviewing the following description of the present disclosure, along with the accompanying figures wherein like reference numerals refer to like parts.

### BRIEF DESCRIPTION OF THE DRAWINGS

The accompanying drawings illustrate the embodiments of systems, methods, and other aspects of the disclosure. A person with ordinary skill in the art will appreciate that the illustrated element boundaries (e.g., boxes, groups of boxes, or other shapes) in the figures represent an example of the boundaries. In some examples, one element may be designed as multiple elements, or multiple elements may be designed as one element. In some examples, an element shown as an internal component of one element may be implemented as an external component in another and vice versa. Furthermore, the elements may not be drawn to scale.

Various embodiments are described in accordance with the appended drawings, which are provided to illustrate and not limit the scope of the claimed subject matter wherein similar designations denote similar elements, and in which:

FIG. 1 illustrates a block diagram of the cardiac health assessment system, in accordance with embodiments of the claimed subject matter.

FIG. 2 illustrates a perspective view of the HED placed against the body or chest of a user, in accordance with embodiments of the claimed subject matter.

FIG. 3 illustrates a perspective view of a wearable device worn by the user to obtain physiological data of the user and transmit it to the HED over a network, in accordance with embodiments of the claimed subject matter.

FIG. 4 illustrates a flowchart of the method for cardiac health assessment, in accordance with embodiments of the claimed subject matter.

### DETAILED DESCRIPTION

The present description is best understood with reference to the detailed figures and description set forth herein. Various embodiments of the present systems and methods have been discussed with reference to the figures. However, those skilled in the art will readily appreciate that the detailed description provided herein with respect to the figures are merely for explanatory purposes, as the present systems and methods may extend beyond the described embodiments. For instance, the teachings presented and the needs of a particular application may yield multiple alternative and suitable approaches to implement the functionality of any detail of the present systems and methods described herein. Therefore, any approach to implement the disclosed systems and methods may extend beyond certain implementation choices in the following embodiments.

According to the described embodiments, the methods may be implemented by performing or completing manually, automatically, and/or a combination of thereof. The term “method” refers to manners, means, techniques, and procedures for accomplishing any task including, but not limited to, those manners, means, techniques, and procedures either known to a person skilled in the art or readily developed from existing manners, means, techniques and procedures by practitioners of the art to which the present invention belongs. A person skilled in the art will envision many other possible variations within the scope of the systems and methods described herein.

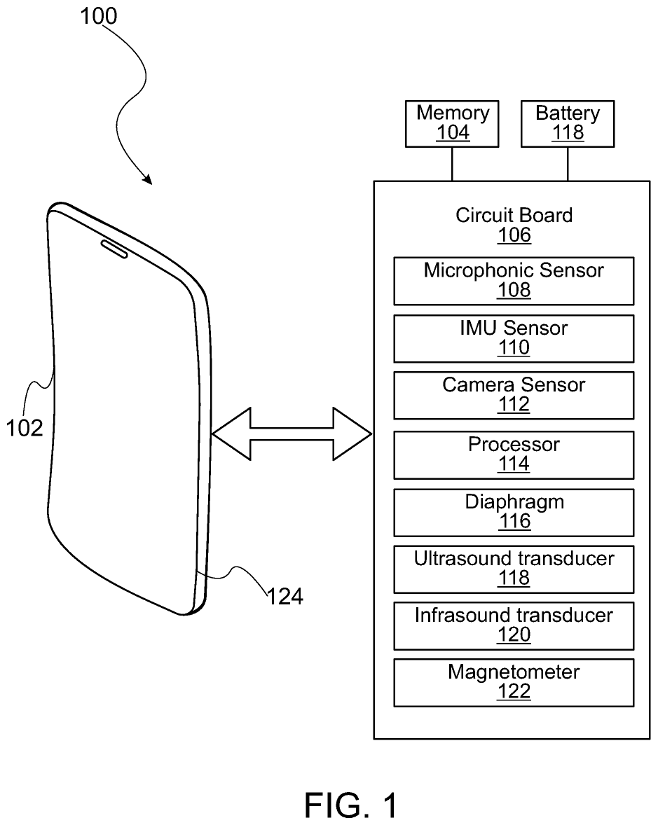

FIG. 1 illustrates a block diagram of the cardiac health assessment system 100, in accordance with at least one embodiment. The cardiac health assessment system 100 includes a memory 104, a circuit board 106, and a touchscreen controller 124 that are integrated into a HED 102. In an embodiment, the HED 102 has a shape adapted to fit firmly on the user's chest. The shape of the HED 102 may also be any suitable shape or it may be constructed so that the shape can be adjustable including being adjusted by the user to a user's shape. In some embodiments, the shape of the HED 102 is bent or curved so that it can fit optimally on the patient's chest. The HED 102 may be similar in shape and function to a smartphone, a mobile device, a phablet, a tablet, and the like. The memory 104 stores a classification model, a regression model, and a plurality of instructions pertaining to a cardiac monitoring application.

In an embodiment, the plurality of instructions pertaining to a cardiac monitoring application may comprise but are not limited to information regarding how and when to execute the classification and regression models, what questions and how the user should be prompted with these questions, information regarding the detection of whether or not the HED has been correctly placed by the user, the prompts given to the user so that the user can log in with their credentials, information regarding ensuring that there are no cyber security risks present, and information regarding enabling data transmission and enabling connectivity with the wearable device coupled to the system. These instructions may take several forms, they may be simple decision rules and/or they may be adaptive to the use patterns of the user to ensure robustness across different users with different interpretations of the information that may be presented in the cardiac monitoring application.

In an embodiment, the cardiac monitoring application is based on one or more operating systems, for example operating systems such as Amazon Fire®, One UI®, Librem®, EMUI®, Android®, and iOS®. The cardiac health assessment system 100 allows the user to register with the cardiac monitoring application configured to operate within the HED 102. In an embodiment, the user may be required to answer a short questionnaire about their health. In some embodiments requested, information may be requested but not required from the user. A user may include a patient, a patient using the cardiac monitoring application using the HED 102 with their own body, and/or any other person such as a healthcare professional using the HED, for example using the HED to execute the functions of the embodiments with a patient.

The memory 104 may be a non-volatile memory or a volatile memory or any combination of these types of memory. Examples of non-volatile memory may include, but are not limited to flash memory, a Read Only Memory (ROM), a Programmable ROM (PROM), Erasable PROM (EPROM), and Electrically EPROM (EEPROM) memory. Examples of volatile memory may include but are not limited to Dynamic Random-Access Memory (DRAM), and Static Random-Access memory (SRAM).

The circuit board 106 includes a microphonic sensor 108, an Inertial Measurement Unit (IMU) sensor 110, a camera sensor 112, and a processor 114. In an embodiment, the circuit board 106 is referred to as a printed wiring board, printed wiring card, or a printed circuit board (PCB) that mechanically supports and electrically connects electrical or electronic components of the embodiments using conductive tracks, pads, and other features etched from one or more sheet layers of copper laminated onto and/or between sheet layers of a non-conductive substrate. The microphonic sensor 108 captures cardiac sound wave signals indicative of the cardiac health of a user and/or a patient. The IMU sensor 110 captures seismic signals indicative of the cardiac health of the user. The camera sensor 112 enables visual data collection of tissue and photoplethysmography. In an embodiment, the camera sensor 112 is a phone camera image sensor such as a CMOS image sensor.

The processor 114 is configured to: execute the plurality of instructions about the cardiac monitoring application; display one or more commands so that the HED 102 can be positioned against the chest of the user, detect an abnormal heart activity arising from a plurality of parameters by deploying the classification model; and estimate intracardiac pressure by deploying the regression model. In an embodiment, ejection fraction, cardiac output and blood pressure may be estimated by deploying the regression model. In an embodiment, the classification model, and the regression model are trained by using intracardiac pressure data measured from a catheter and/or an invasive pressure sensor which are considered the gold standard in the industry for measuring such types of data. According to several embodiments, gold standard ejection fraction data may comprise data obtained from an ultrasound analysis and/or cardiovascular magnetic resonance (CMR) that is used to train the classification model and regression model. According to many embodiments, gold standard cardiac output data may comprise data obtained from a Pulmonary Artery catheter-based thermodilution. According to many embodiments, gold standard blood pressure data may comprise data obtained from a sphygmomanometer and/or an arterial line.

Memory 104 is configured to register the user over the cardiac monitoring application by receiving one or more credentials from the user for providing access to the cardiac monitoring application. Examples of the credentials, include but are not limited to, a username, password, age, gender, phone number, email address, location, as well as any other suitable information. In several embodiments, the cardiac monitoring application is commercialized as a software application or a mobile application, a web application for cardiac health assessment or any combination of these.

The processor 114 may include at least one data processor for executing program components for executing user- or system-generated requests. Processor 114 may include specialized processing units such as integrated system (bus) controllers, memory management control units, floating point units, graphics processing units, digital signal processing units, etc. Processor 114 may include a microprocessor, such as AMD® ATHLON® microprocessor, DURON® microprocessor OR OPTERON® microprocessor, ARM's application, embedded or secure processors, IBM® POWERPC®, INTEL'S CORE® processor, ITANIUM® processor, XEON® processor, CELERON® processor or other line of processors, etc. Processor 114 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), and the like.

Processor 114 may be disposed of in communication with one or more input/output (I/O) devices via an I/O interface. I/O interface may employ communication protocols/methods such as, without limitation, audio, analog, digital, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), and the like.

In an embodiment, the processor 114 triggers a message transmission to a computing device, for instance a computing device used by a healthcare professional, upon detection of high severity of heart disease based on the cardiac signals captured by the HED. Examples of computing device include but are not limited to a laptop, a desktop, smartphone, a server, and any combination of these.

In several embodiments, the processor 114 identifies a plurality of unique physiological markers of the user based on historical cardiac signals captured by the HED 102. In an embodiment, processor 114 is configured to present a plurality of instructions regarding the management of the user's disease. The touchscreen controller 124 displays cardiac diagnostic information received from the HED 102 that is derived from the cardiac sensor signal data.

In an embodiment, the touchscreen controller 124 is a capacitive touch screen that uses the conductive touch of a human finger or a specialized input device. The touchscreen controller 124 provides a User Interface (UI) used by the user or an administrator to initiate a request to view the data assessed by the cardiac health assessment system and provide various inputs to the cardiac health assessment system 100. In many of the embodiments, the UI also known as a Graphic User Interface (GUI) is a convenient interface for accessing the information related to the status of the user's heart health. The touchscreen controller 124 may be operated by a display driver such as an LCD driver or a LED driver. In many embodiments, the touchscreen controller 124 includes an ASIC (application-specific integrated circuit) a digital signal processor (DSP) and/or any other suitable technology known to those skilled in the art.

In many embodiments, the plurality of parameters that may be sensed and/or monitored includes hypertension, atrial fibrillation, ischemic cardiomyopathy, aortic stenosis, aortic regurgitation, mitral stenosis, and/or mitral regurgitation. It is well-known that hypertension and atrial fibrillation can be detected using visual based analysis including but not limited to photoplethysmography and/or analyzing acoustic patterns related to irregular heartbeats and/or velocity of blood-flow and/or patterns related to pulse transmit time. It is further well known that ischemic cardiomyopathy, aortic stenosis, aortic regurgitation, mitral stenosis, regurgitation can result in abnormal seismic and/or acoustic activity that can be detected using inertial measurement units, microphonic sensors and/or other soundwave-based technologies such as ultrasound transceivers. These abnormal seismic and/or acoustic activities may results from arterial blockages and/or abnormal sounds relating to excess blood-flow from cardiac leakages between chambers in the heart. In some embodiments, the cardiac health assessment system 100 includes a battery 118 configured to supply electrical power to the circuit board 106. According to some embodiments, battery 118 is based on Lithium Polymer (Li-Poly) and Lithium-Ion (Li-Ion). In some embodiments, the battery 118 is operated by a power management integrated circuit such as power MOSFETs.

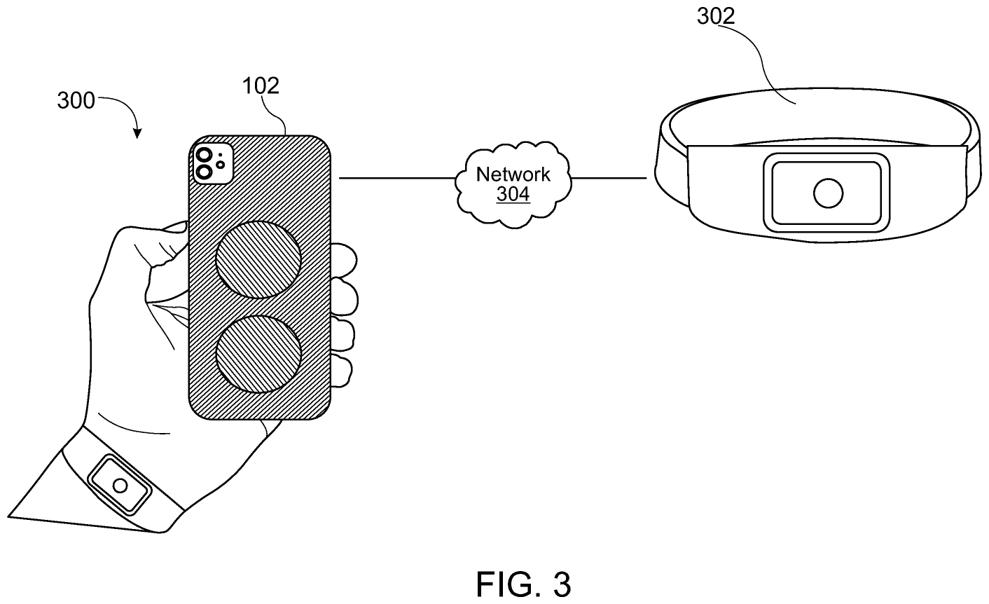

FIG. 3 illustrates a system 300 including a wearable device 302 worn by the user to obtain physiological data of the user so that the physiological data can be transmitted to the HED 102 over a network in accordance with several embodiments. Examples of a wearable device 302 include but are not limited to a watch, a strap, a smartwatch, one or more pieces of smart jewelry such as rings, a wristband, a torso strap, or any other suitable device that can be worn or attached to a user and which can be used to sense data that can be transmitted to the HED 102. In some embodiments, the wearable device 302 includes one or more body-mounted sensors that monitor and transmit biological data for healthcare or other monitoring purposes. In many embodiments, the wearable device 302 can act as a fitness tracker that monitors the physical activity and vital signs of the users. According to one embodiment, circuit board 106 includes a transceiver to establish a communication with the network 304. Network 304 may be one or more wired or wireless networks, or any combination of wired and wireless networks, and the examples may include but are not limited to the internet, a Wireless Local Area Network (WLAN), a Wi-Fi network, a Long Term Evolution (LTE) network, a Worldwide Interoperability for Microwave Access (WiMAX) network, and a General Packet Radio Service (GPRS) network.

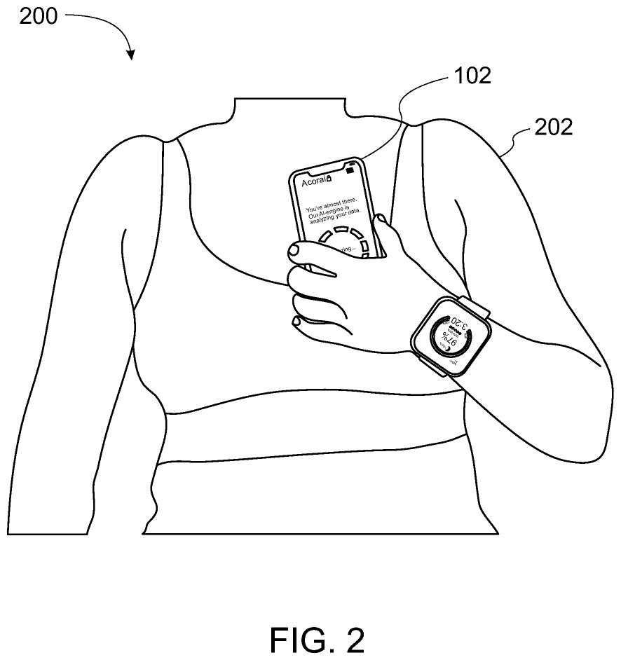

In an embodiment, the circuit board 106 includes a diaphragm 116 to enhance the cardiac audio signals captured by the microphonic sensor. FIG. 2 illustrates a perspective view 200 of the HED 102 placed against the body or chest of a user 202 in accordance with several embodiments. In many embodiments, the circuit board 106 includes an ultrasound transducer 118 for transmitting high-frequency waves into the body of the user to detect data relating to many physiological processes including, but not limited to, intracardiac blood pressure and blood flow and return sound wave data. This data can then be analyzed with the classification model and the regression model simultaneously with the detection, at a later time, or both simultaneously and a later time. In some embodiments, the circuit board 106 includes an infrasound transducer 120 for transmitting low-frequency waves into the body of the user to detect data relating to physiological processes and return wave data which can be analyzed with the classification model and the regression model simultaneously with the detection, at a later time, or both simultaneously and a later time. In many embodiments, the circuit board 106 includes a magnetometer 122 for detecting abnormal or normal traces of ferromagnetic levels in the blood of the user. In many of the embodiments, the classification model and the regression model utilize machine learning (ML) to analyze the cardiac health of the user. Other models may also be used with or without ML and other data may be used in combination with the derived data.

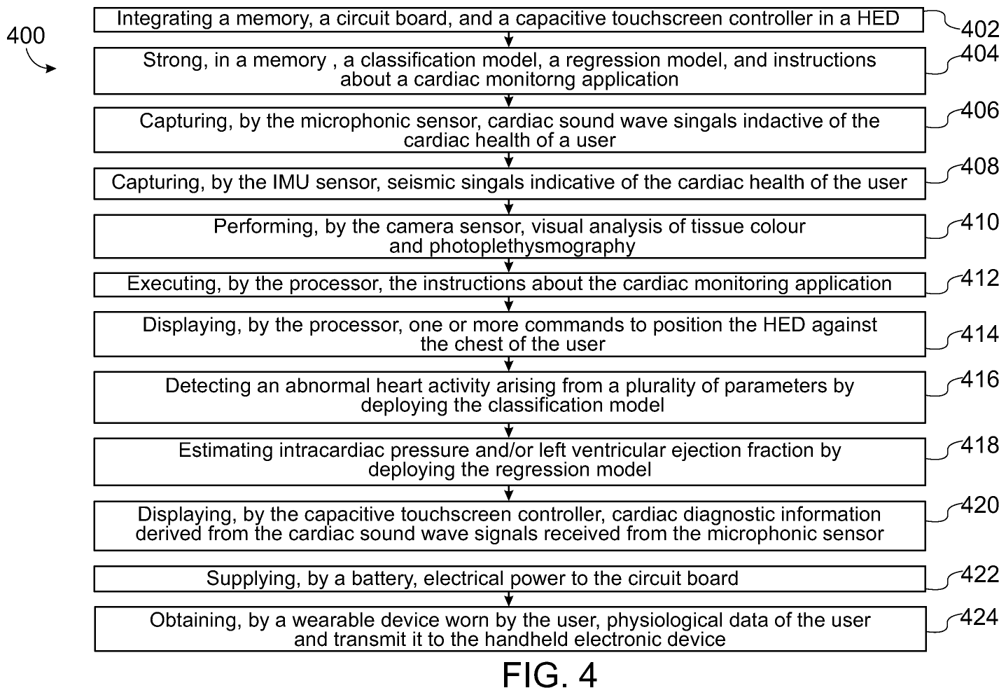

FIG. 4 illustrates a flowchart 400 of the method for cardiac health assessment, in accordance with embodiments of the claimed subject matter. The method includes step 402 of integrating a memory, a circuit board, and a touchscreen controller in a HED. In these embodiments, the touchscreen may comprise resistive and capacitive characteristics and may use indium tin oxide (ITO) sensors. Also in these embodiments, the HED has a shape adapted to fit firmly on the user's chest. The method includes step 402 of storing, in a memory, a classification model, a regression model, and a plurality of instructions about a cardiac monitoring application. The circuit board includes a microphonic sensor, an IMU sensor, a camera sensor, a processor, a diaphragm, an ultrasound transducer, an infrasound transducer, and a magnetometer. The method includes step 406 of capturing by the microphonic sensor cardiac sound wave signals indicative of the cardiac health of a user. The method includes step 408 of using the IMU sensor to capture seismic signals indicative of the cardiac health of the user. The method includes step 410 of using the camera sensor to perform visual data collection and/or photoplethysmography. The method includes step 412 of using the processor to execute the cardiac monitoring application instructions. In many embodiments, the cardiac monitoring application may be based on one or more of the following operating systems: Amazon Fire®, One UI®, Librem®, EMUI®, Android®, and iOS®. The method includes step 414 of using the processor to display to the user one or more commands to position the HED against the chest of the user. The method includes step 416 of detecting, by the processor, an abnormal heart activity arising from a plurality of parameters by deploying the classification model. In many embodiments, the plurality of parameters may include indications of hypertension, ischemic cardiomyopathy, aortic stenosis, aortic regurgitation, mitral stenosis, and/or mitral regurgitation. The method includes step 418 of estimating, by the processor, intracardiac pressure and/or ejection fraction by deploying the regression model. In some embodiments, the classification model and/or the regression model are trained by using intracardiac pressure data measured from a catheter and/or an invasive sensor (sensors that are recognized as providing gold standard data.) The method includes step 420 of displaying, by the touchscreen controller, cardiac diagnostic information derived from the cardiac sound wave signals received from the microphonic sensor. The method further comprises step 422 of supplying, by a battery, electrical power to the circuit board. In some embodiments, the processor triggers a message transmission to a computing device of a healthcare professional (or any other device capable of receiving messages) upon a detection of high severity of heart disease based on the cardiac sound wave signals captured by the microphonic sensor. In many embodiments, the processor identifies a plurality of unique physiological markers of the user based on historical cardiac sound wave signals captured by the microphonic sensor. The historical cardiac sound wave signals may have been previously captured by the microphonic sensor or the historical cardiac sound wave signals may have been captured by another microphonic sensor, another sensor or they may be derived from stored data contained in one or more databases. The stored data may be data that was manually entered or it may be data automatically captured by one or more other manual or automatic methods, for instance manually entering historical data by a health professional or using data captured over a range of patients and/or users at one time or over a period of time. In many embodiments, the processor is configured to present a plurality of instructions regarding the management of the user's disease.

The method further comprises step 424 of obtaining, by a wearable device worn by the user, physiological data of the user and transmit it to the HED over a network. In many embodiments, the diaphragm enhances the cardiac audio signals captured by the microphonic sensor. Any other suitable signal enhancer known to those skilled in the art may be used to enhance the one or more signals in the embodiments. In many embodiments, the ultrasound transducer transmits high-frequency waves into the body of the user to deflect a plurality of physiological processes comprising intracardiac blood pressure and blood flow and return sound wave data to be analyzed with the classification model and the regression model. In some embodiments, the infrasound transducer transmits low-frequency waves into the body of the user to deflect the physiological processes and return wave data to be analyzed with the classification model and the regression model. In an embodiment, the magnetometer detects abnormal traces of ferromagnetic levels in the blood of the user.

Thus, embodiments of the cardiac health assessment systems and methods provide a real-time diagnostic mechanism for heart disease and heart failure. The cardiac monitoring application incorporates reminders, nudges, and notifications to help the heart patient to track the risk of heart diseases and progression of heart failure.

Accordingly, one advantage of the claimed subject matter is that it provides a telehealth mechanism to facilitate the user to interact with a clinician or get on a video call with the clinician.

Another advantage of the claimed subject matter is that the use of telehealth mechanism allows the user to interact with a medical chatbot.

Another advantage of the claimed subject matter is that the telehealth mechanism allows for remote patient monitoring and management of heart diseases and heart failure.

Another advantage of the claimed subject matter is that the telehealth mechanism allows for treatment and medication adjustments based on the results assessed by the HED.

In the foregoing specification, embodiments of the claimed subject matter have been described with reference to numerous specific details that may vary from implementation to implementation. The specification and drawings are, accordingly, to be regarded in an illustrative rather than a restrictive sense. The embodiments are only limited by the scope of the claimed subject matter.

What is claimed is:

1. A cardiac health assessment system, comprising:
a handheld electronic device (HED) comprising:
a memory configured to store a classification model and/or a regression model, and a plurality of instructions for a cardiac monitoring application;
a circuit board;
a microphonic sensor in communication with the circuit board configured to capture cardiac sound wave signals indicative of the cardiac health of a user;
an Inertial Measurement Unit sensor in communication with the circuit board for capturing seismic signals indicative of the cardiac health of the user;
a camera sensor in communication with the circuit board to enable visual analysis of the user's tissue;
a processor in communication with the circuit board configured to:
execute the plurality of instructions for the cardiac monitoring application;
display one or more commands to position the HED against the chest of the user;
estimate intracardiac pressure by deploying the regression model; and
a touchscreen controller in communication with the circuit board to display cardiac health information of the user.

2. The cardiac health assessment system as claimed in claim 1 further comprising a battery configured to supply electrical power to the HED.

3. The cardiac health assessment system of claim 1, wherein the HED has a curved shape adapted to fit firmly on the user's chest.

4. The cardiac health assessment system of claim 1, wherein the processor is configured to detect an abnormal heart activity arising from a plurality of parameters by deploying the classification model.

5. The cardiac health assessment system of claim 4, wherein the classification model has been trained to detect one or more of the following health conditions: hypertension, ischemic cardiomyopathy, heart arrythmias, aortic stenosis, aortic regurgitation, mitral stenosis and mitral regurgitation.

6. The cardiac health assessment system of claim 1, wherein the processor triggers a message transmission comprising of the user's data to another computing device.

7. The cardiac health assessment system of claim 1, wherein the processor identifies a plurality of unique physiological identification markers of the user based on the cardiac sound wave signals captured by the microphonic sensor.

8. The cardiac health assessment system of claim 1, wherein the processor is configured to present a plurality of instructions regarding the management of the user's disease.

9. The cardiac health assessment system of claim 1, wherein the classification model and the regression model are trained using intracardiac pressure data measured from one or more of the following: a catheter and an invasive sensor.

10. The cardiac health assessment system of claim 1, wherein the cardiac monitoring application is based on one or more of the following operating systems: Amazon Fire®, One UI®, Librem®, EMUI®, Android®, and iOS®.

11. The cardiac health assessment system of claim 1, further comprising a wearable device worn by the user to obtain physiological data of the user and transmit it to the HED over a network.

12. The cardiac health assessment system of claim 1, wherein the HED further comprises a diaphragm to enhance the cardiac audio signals captured by the microphonic sensor.

13. The cardiac health assessment system of claim 1, further comprising a soundwave transducer in communication with the circuit board for transmitting soundwaves into the body of the user to detect a plurality of physiological processes comprising intracardiac pressure and heart muscles.

14. The cardiac health assessment system of claim 1, further comprising a magnetometer in communication with the circuit board to detect abnormal traces of ferromagnetic levels in the blood of the user.

15. The cardiac health assessment system of claim 1, wherein the processor is further configured to display one or more commands for positioning the HED against the thoracic cage of the user.

16. The cardiac health assessment system of claim 1, wherein the processor is further configured to detect abnormal pulmonary health activity arising from a plurality of parameters by deploying a pulmonary disease classification model.

17. The cardiac health assessment system of claim 1, wherein the processor is further configured to display one or more commands for positioning the HED against a leg of the user.

18. The cardiac health assessment system of claim 1, wherein the processor is further configured to detect abnormal thrombotic activity arising from a plurality of parameters by deploying a deep vein thrombosis classification model.

19. The cardiac health assessment system of claim 1, wherein the processor is further configured to estimate lung fluid levels arising from a plurality of parameters by deploying a lung fluid estimation model.

20. The cardiac health assessment system of claim 1, further comprising a temperature sensor in communication with the circuit board for measuring one or more temperatures of the user.

21. The cardiac health assessment system of claim 1, wherein the processor is further configured to estimate patient hospitalization risk by deploying a patient risk stratification model.

22. A method for cardiac health assessment, comprising:
integrating a memory, a circuit board, and a touchscreen controller in a handheld electronic device (HED);
storing, in a memory, a classification model, a regression model, and a plurality of instructions for a cardiac monitoring application; wherein the circuit board is connected to a microphonic sensor, an Inertial Measurement Unit (IMU) sensor, a camera sensor, and a processor;
capturing, by the microphonic sensor, cardiac sound wave signals indicative of the cardiac health of a user;
capturing, by the IMU sensor, seismic signals indicative of the cardiac health of the user;
performing, by the camera sensor, visual data collection of tissue and photoplethysmography;
executing, by the processor, the instructions pertaining to the cardiac monitoring application;
displaying, by the processor, one or more commands to aid in the positioning of the HED against the chest of the user;
estimating, by the processor, intracardiac pressure by deploying the regression model; and
displaying, by the touchscreen controller, cardiac health information derived from the classification model and the regression model.

23. The method as claimed in claim 22 further comprising a step of supplying, by a battery, electrical power to the circuit board.

24. The method as claimed in claim 22, wherein the HED has a shape adapted to fit firmly on the user's chest.

25. The method as claimed in claim 22, wherein the processor triggers a message transmission to another computing device upon detection of a high severity of heart disease based on the estimation from the regression model.

26. The method as claimed in claim 22, wherein the method comprises detecting, by the processor, an abnormal heart activity arising from a plurality of parameters by deploying the classification model.

27. The method as claimed in claim 26, wherein the plurality of parameters comprise one or more of the following: hypertension, heart arrythmias, ischemic cardiomyopathy, aortic stenosis, aortic regurgitation, mitral stenosis, and mitral regurgitation.

28. The method as claimed in claim 22, wherein the processor identifies a plurality of unique physiological markers of the user based on cardiac sound wave signals captured by the microphonic sensor.

29. The method as claimed in claim 22, wherein the processor is configured to present a plurality of instructions regarding the management of the user's disease.

30. The method as claimed in claim 22, wherein the classification model and the regression model are trained by using intracardiac pressure data measured from one or more of the following: a catheter and an invasive sensor.

31. The method as claimed in claim 22, wherein the cardiac monitoring application is based on one or more of the following operating systems: Amazon Fire®, One UI®, Librem®, EMUI®, Android®, and iOS®.

32. The method as claimed in claim 22 further comprising a step of obtaining, by a wearable device worn by the user, physiological data of the user and transmitting it to the HED over a network.

33. The method as claimed in claim 22, wherein the cardiac audio signals captured by the microphonic sensor are enhanced by a diaphragm.

34. The method as claimed in claim 22, further comprising the step of transmitting sound waves into the body of the user to detect a plurality of physiological processes indicating intracardiac blood pressure and blood flow with the use of a sound transducer connected to the circuit board.

35. The method as claimed in claim 22, further comprising the step of detecting abnormal traces of ferromagnetic levels in the blood of the user using a magnetometer connected to the circuit board.

36. The method as claimed in claim 22, wherein the one or more commands displayed by the processor to aid in the positioning of the HED are used to aid the user in positioning the HED against the thoracic cage of the user.

37. The method as claimed in claim 22, further comprising the step of using the processor to detect abnormal pulmonary health activity arising from a plurality of parameters by deploying a pulmonary disease classification model

38. The method as claimed in claim 22, wherein the one or more commands displayed by the processor to aid in the positioning of the HED are used to aid the user in positioning the HED against the leg of the user.

39. The method as claimed in claim 22, further comprising the step of using the processor to detect abnormal thrombotic activity arising from a plurality of parameters by deploying a deep vein thrombosis classification model.

40. The method as claimed in claim 22, further comprising the step of using the processor to estimate lung fluid levels arising from a plurality of parameters by deploying a lung fluid estimation model.

41. The method as claimed in claim 22, further comprising the step of measuring one or more temperatures of the user with the use of a temperature sensor connected to the circuit board.

42. The method as claimed in claim 22, further comprising the step of using the processor to estimate patient hospitalization risk by deploying a patient risk stratification model.
